(19) United States
(12) Patent Application Publication
(10) Pub. No.: US 2025/0360016 A1
(43) Pub. Date: Nov. 27, 2025

(54) ADJUSTABLE TOE SPACER

(71) Applicant: Zentoes LLC, Bellingham, WA (US)

(72) Inventor: Noa Strizover, Scottsdale, AZ (US)

(21) Appl. No.: 18/674,834

(22) Filed: May 25, 2024

(51) Int. Cl. A61F 5/01

(52) U.S. Cl. CPC A61F 5/019

(57) ABSTRACT

The present disclosure relates to an adjustable toe spacer that helps restore the natural shape of the foot during use by a user. The adjustable toe spacer includes a pair of base walls and multiple spacer elements. The spacer elements and the pair of base walls are connected to define holes that are dimensioned to receive the toes of the user. The shape and the material of the adjustable toe spacer provide a comfortable fit for the toes of the user. Further, the adjustable toe spacer provides an option of removably inserting accessories such as wedge insert(s) in respective slots of the adjustable toe spacer to enable easy and quick size adjustment of the adjustable toe spacer.

### TECHNICAL FIELD

The present invention relates to the field of toe spacer for use on the foot of a user and more particularly, the present invention relates to an adjustable toe spacer.

### BACKGROUND

Many individuals experience toe crowding or overcrowding, or the phenomena of the toes tending to rest in a more crowded position, even when not within a shoe. Toe crowding is typically a result of wearing shoes with a narrow toe box.

Toe crowding can lead to toe overlapping, with toes resting atop one another in an overlapped position, which increases friction and causes discomfort. Another common result of overcrowding is pinched toes, which occurs when toes rub against each other excessively, resulting in the development of soft corns between the toes.

Hammertoe is another disorder that can develop due to overcrowding. A hammertoe acquires a crooked or clawed shape. Due to various factors such as inadequate shoes or genetic reasons that increase the likelihood of developing such injuries, toe overcrowding usually leads to the development of muscle or ligament imbalances that create increased pressure on the toes' joints; this causes them to bend and potentially become held in this position.

Another disorder that can develop due to overcrowded toes is known as Morton's neuroma. Women account for the vast majority of cases of Morton's neuroma due to footwear problems. Using high-heeled, narrow, and pointy shoes can place substantial pressure on the forefoot, particularly on the toes. An increased pressure on the forefoot can place an increased pressure on the forefoot's nerves, causing entrapments or other mechanical traumas. Local blood flow may also be affected, leading to injury due to low oxygen supply. As a consequence, a nerve may become deformed and inflammation may develop, ultimately leading to the formation of a neuroma.

Various toe spacers exist in the prior art that address toe crowding. The existing toe spacers create space between the toes, helping restore the natural shape of the foot during use. However, the existing toe spacers are often very firm and made of a restrictive material which makes them uncomfortable in use. Further, the foot sizes and foot types of various persons are different depending on their age, gender, genetics, and so on, thus requiring an adjustable toe spacer that better suits the requirements of the particular user. Additionally, while there are adjustable toe spacers in the market, those do not come with the proper and necessary accessories to enable easy and quick adjustment (customization) for the adjustable toe spacers.

For instance, U.S. Pat. No. 2,595,640A discloses a toe spacing device adapted to be inserted between the toes of a foot of a human being for separating the toes to facilitate the application of nail polish to the toenails. The toe spacing device includes a plurality of spacing members or wedges which are flexibly held in assembled relationship for insertion between the toes of a foot to provide for separation of the toes during application of nail polish and during drying thereof. The toes of the human foot normally tend to lie quite close to each other and this relationship of the toes is further promoted by the use of narrow-width shoes. Thus when polish in liquid form is to be applied to the toe nails in a manicuring operation there is a tendency for the polish carried by the nail of one toe to smear or to be deposited on an adjacent toe before the polish has had an opportunity to dry or set sufficiently.

US20070027011A1 discloses an exercise tool that is specifically devised as a toe stretcher to align, separate, and stretch toes. The toe stretcher comprises a frame with a separator for separating a plurality of toes. The frame further includes holes or indentations for placement of the toes. Additionally, the frame is flexible, allowing a user to easily manipulate the toe stretcher and place it on the user's toes, effectively separating and stretching toes. Furthermore, the toe stretcher may be attached with footwear, allowing a user to not only stretch toes, but also use the toe stretcher as a footwear.

The existing solutions related to toe spacers are inefficient and ineffective, rigid, uncomfortable in use, and do not allow easy and quick size adjustments. There is a need for an effective and efficient solution that solves the aforementioned problem by providing a new and improved adjustable toe spacer.

### SUMMARY

An object of the present invention is the provision of an adjustable toe spacer that helps restore the natural shape of the foot during use.

An object of the present invention is the provision of a novel, simple, and efficient adjustable toe spacer capable of being customized according to the requirements of the user by using accessories such as wedge inserts.

An object of the present invention is the provision of a novel, simple, and efficient adjustable toe spacer that can be customized without requiring any special tools.

An object of the present invention is the provision of a novel, simple, and efficient adjustable toe spacer that is latex-free, bisphenol A (BPA) free, and can be easily washed and reused multiple times.

While the way that the present disclosure addresses the disadvantages of the prior art will be discussed in greater detail below, in general, the present disclosure provides an adjustable toe spacer comprising a pair of base walls arranged substantially parallel to each other, a plurality of spacer elements interconnecting the pair of base walls at multiple locations, wherein the plurality of spacer elements and the pair of base walls are connected to define a plurality of holes that are dimensioned to receive a plurality of toes of the user, at least one slot that extends in a spacer selected from the plurality of spacer elements, wherein the slot(s) is configured to removably receive a wedge insert for adjusting spacing between the plurality of toes of user.

In an embodiment, the pair of base walls and the plurality of spacer elements are integrally connected and made of styrene-ethylene-butylene-styrene (SEBS) gel.

In an embodiment, the pair of base walls and the plurality of spacer elements are integrally connected and made of silicone gel.

In an embodiment, the pair of base walls and the plurality of spacer elements are made of a tearable (trimmable) material that is configured to be trimmed thinner for enabling use by a user with an overspread pinky toe.

In an embodiment, the plurality of spacer elements includes a first spacer element measuring 0.35 inch in width, a second spacer element measuring 0.12 inch in width, a third spacer element measuring 0.12 inch in width, and a fourth spacer element measuring 0.28 inch in width.

In an embodiment, the wedge insert is made of Ethylene-vinyl acetate (EVA) foam.

In an embodiment, the wedge insert is substantially triangular-shaped to enable easy insertion and removal in the respective slot(s).

In an embodiment, the plurality of spacer elements are four in number.

In an embodiment, the plurality of holes includes a total of three holes.

In an embodiment, the slot(s) is a through-hole.

In an embodiment, each slot(s) includes a pair of slot openings that are formed in the pair of base walls.

In an embodiment, the plurality of holes is dimensioned to receive an index toe, a middle toe, and a ring toe of the user.

These and other features and advantages of the present invention will become apparent from the detailed description below, in light of the accompanying drawings.

### BRIEF DESCRIPTION OF THE DRAWINGS

A more complete understanding of the present disclosure may be derived by referring to the detailed description and claims when considered in connection with the figures, wherein like reference numerals refer to similar elements throughout the figures, and

FIG. 1 illustrates a front perspective view of an adjustable toe spacer, according to an embodiment of the present invention.

FIG. 2 illustrates a top-sectional view of the adjustable toe spacer of FIG. 1.

FIGS. 3-5 illustrate a top view, a front view, and a back view of a pair of adjustable toe spacers respectively, according to an embodiment of the present invention.

FIG. 6 illustrates a top view of a pair of adjustable toe spacers of FIGS. 3-5 worn by the user, according to an embodiment of the present invention.

FIG. 7 illustrates a top view of a pair of adjustable toe spacers of FIGS. 3-5 worn by the user, wherein a plurality of wedge inserts are removably inserted in slots of the adjustable toe spacer of FIGS. 3-5.

FIG. 8 illustrates a front perspective view of a plurality of wedge inserts of FIG. 7.

### DETAILED DESCRIPTION

The following description is of exemplary embodiments of the invention only, and is not intended to limit the scope, applicability, or configuration of the invention. Rather, the following description is intended to provide a convenient illustration for implementing various embodiments of the invention. As will become apparent, various changes may be made in the function and arrangement of the elements described in these embodiments without departing from the scope of the invention as set forth herein. It should be appreciated that the description herein may be adapted to be employed with alternatively configured devices having different shapes, components, attachment mechanisms and the like and still fall within the scope of the present invention. Thus, the detailed description herein is presented for purposes of illustration only and not for limitation.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

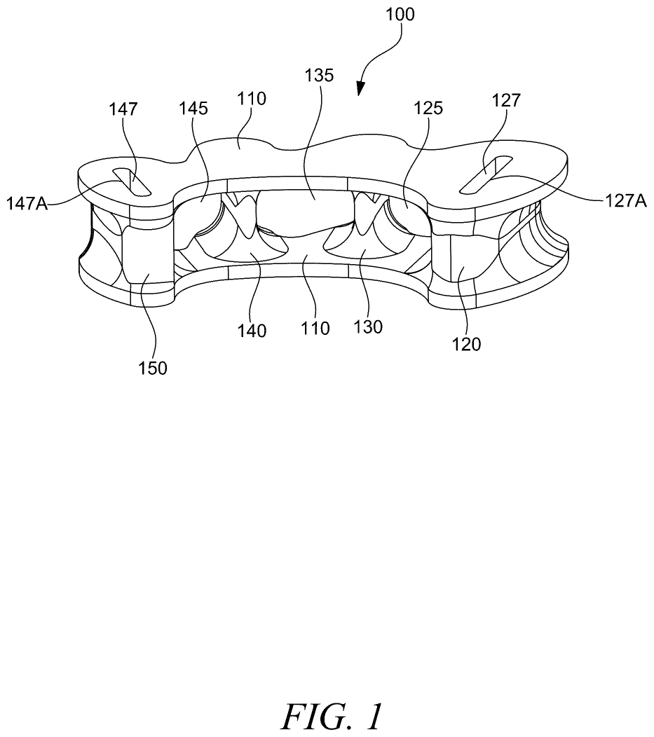

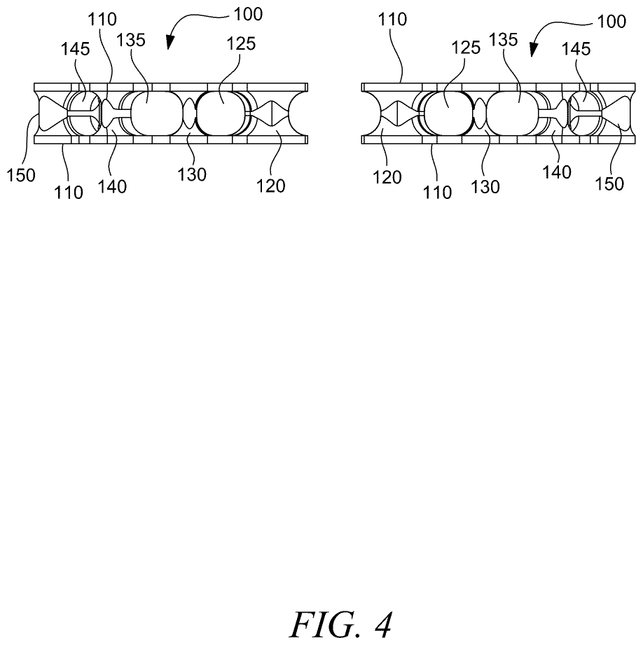

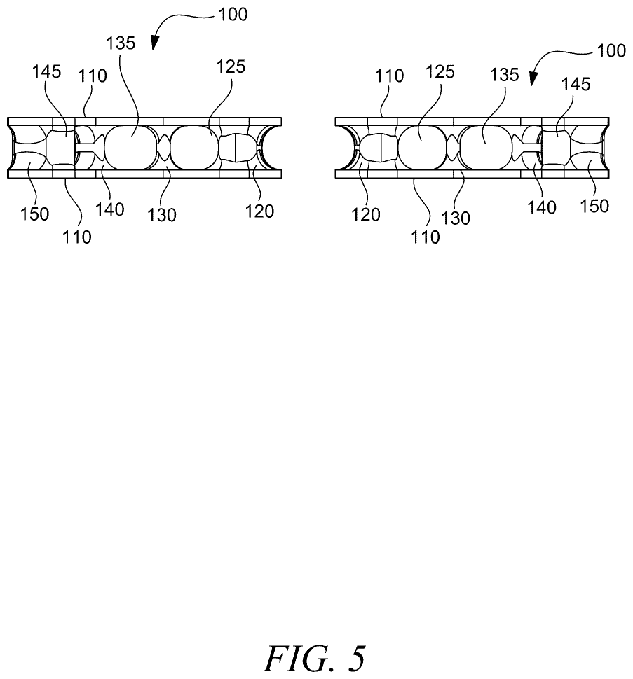

The adjustable toe spacer will now be described with reference to the accompanying drawings, particularly FIGS. 1-8.

Reference is initially made to FIG. 1 that illustrates a front perspective view of an adjustable toe spacer 100, according to an embodiment of the present invention. The adjustable toe spacer 100 is configured to create space between the toes, helping restore the natural shape of the foot during use. The adjustable toe spacer 100 promotes proper toe alignment during use to help relieve pressure, pain, and tension from crowded or overlapping toes, hammertoes, bunions (big toe), and bunionettes (pinky toe). The adjustable toe spacer 100 comprises a pair of base walls 110 arranged substantially parallel to each other. The pair of base walls 110 are substantially identical in shape and size and are curvilinear in shape. A plurality of spacer elements (120, 130, 140, 150) interconnects the pair of base walls 110 at multiple points (positions).

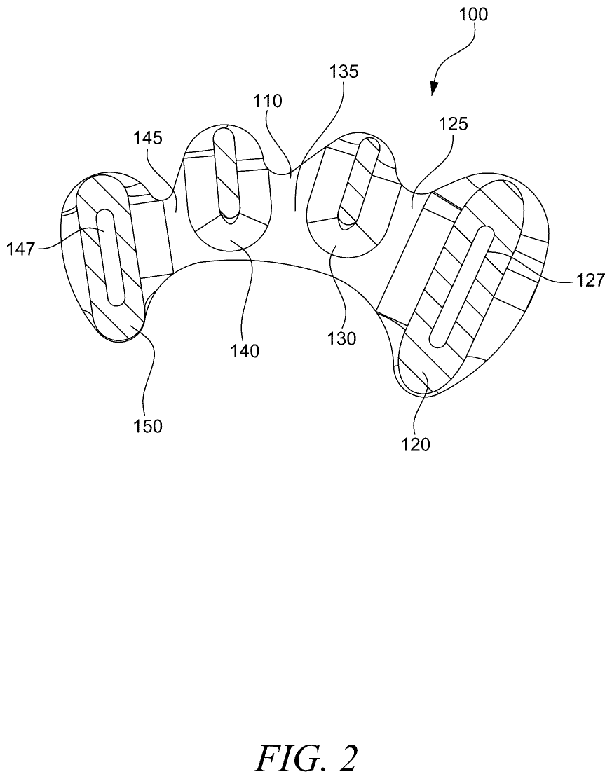

In an embodiment as seen in FIG. 1 and FIG. 2, the total number of spacer elements (120, 130, 140, 150) is four that is a first spacer 120, a second spacer element 130, a third spacer element 140, and a fourth spacer element 150. In an example embodiment, the plurality of spacer elements (120, 130, 140, 150) includes a first spacer element 120 measuring 0.35 inch in width, a second spacer element 130 measuring 0.12 inch in width, a third spacer element 140 measuring 0.12 inch in width, and a fourth spacer element 150 measuring 0.28 inch in width. However, it should be understood that the size of the spacer elements (120, 130, 140, 150) is not a limiting factor of the present invention and the sizing can be adjusted according to the needs of the user.

In another embodiment (not shown in figures), the total number of spacer elements (120, 130, 140, 150) is more than four that is a first spacer 120, a second spacer element 130, a third spacer element 140, and a fourth spacer element 150, a fifth spacer (not shown in figures), and so on.

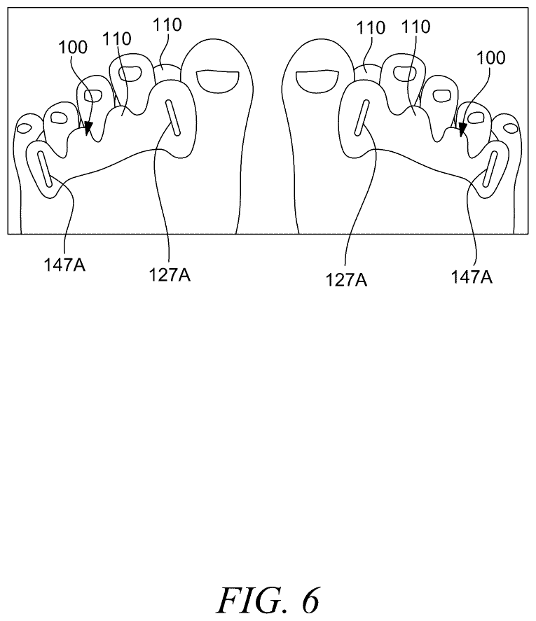

The spacer elements (120, 130, 140, 150) and the pair of base walls 110 are connected to define (form) a plurality of holes (125, 135, 145) that are dimensioned to receive a plurality of toes of the user. As seen in FIG. 1 and FIG. 6, the plurality of holes (125, 135, 145) includes a total of three holes that includes a first hole 125 dimensioned to receive an index toe, a second hole 135 dimensioned to receive a middle toe, and a third hole 145 dimensioned to receive ring toe of the user.

In another embodiment (not shown in figures), the plurality of holes (125, 135, 145) includes more than three holes. In an exemplary embodiment (not shown in figures), the holes (125, 135, 145) include a first hole 125 dimensioned to receive an index toe, a second hole 135 dimensioned to receive a middle toe, a third hole 145 dimensioned to receive ring toe of the user, a fourth hole (not shown in figures) dimensioned to receive big toe of the user and a fifth hole (not shown in figures) dimensioned to receive pinky toe of the user.

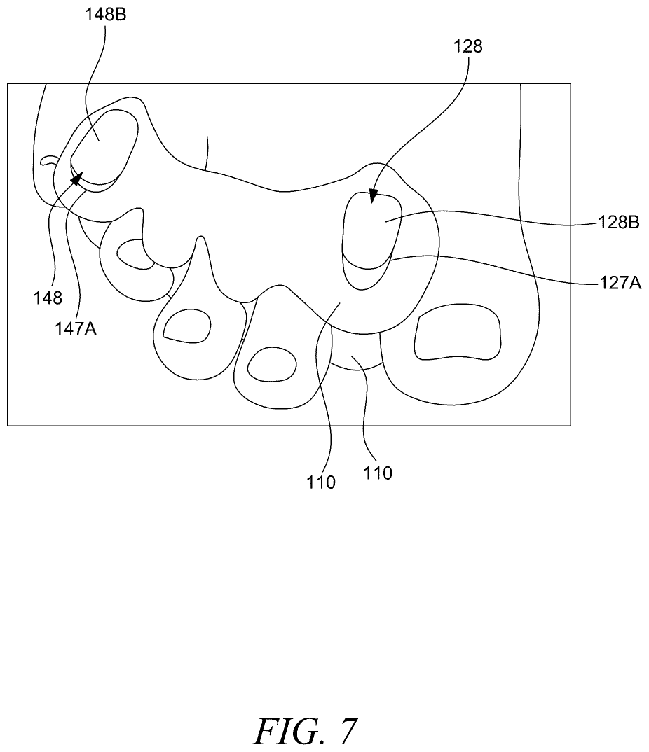

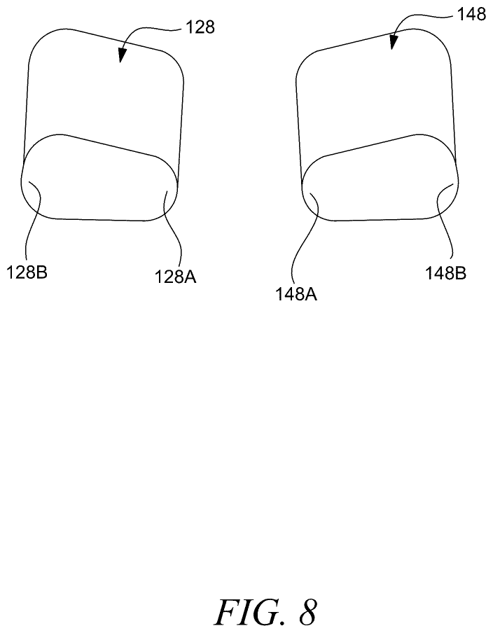

A plurality of slots (127, 147) extends in a spacer element (120, 130, 140, 150), wherein the slots(s) (127, 147) are configured to removably receive accessories in the form of wedge inserts (128, 148) (FIGS. 7-8) for adjusting the spacing between the toes of the user. As seen in FIG. 1 and FIG. 6, the slots (127, 147) include a first slot 127 that extends in a first spacer element 120, and a second slot 147 that extends in a fourth spacer element 150. Each slot (127, 147) is a through-hole and includes a pair of slot openings (127A, 147A) and (127B, 127B) disposed on the opposite ends of the slot (127, 147), wherein the pair of slot openings (127A, 147A) and (127B, 127B) are formed in the pair of base walls 110.

In another embodiment (not shown in figures), the slots (127, 147) are more or less than two in number. In an exemplary embodiment (not shown in figures), the slots include a first slot 127 that extends in a first spacer element 120, a second slot 147 that extends in a fourth spacer element 150, a third slot (not shown in figures) that extends in a second spacer element 130 and a fourth slot (not shown in figures) that extends in a third spacer element 140. In another exemplary embodiment (not shown in figures), the slots include a first slot 127 that extends in a first spacer element 120. In another exemplary embodiment (not shown in figures), the slots include a second slot 147 that extends in the fourth spacer element 150. The advantage of having more or less than two slots (127, 147) is that the user can increase/decrease (customize) the spacing between the toes according to their requirement by inserting wedge inserts (128, 148) in more or less than two slots (127, 147).

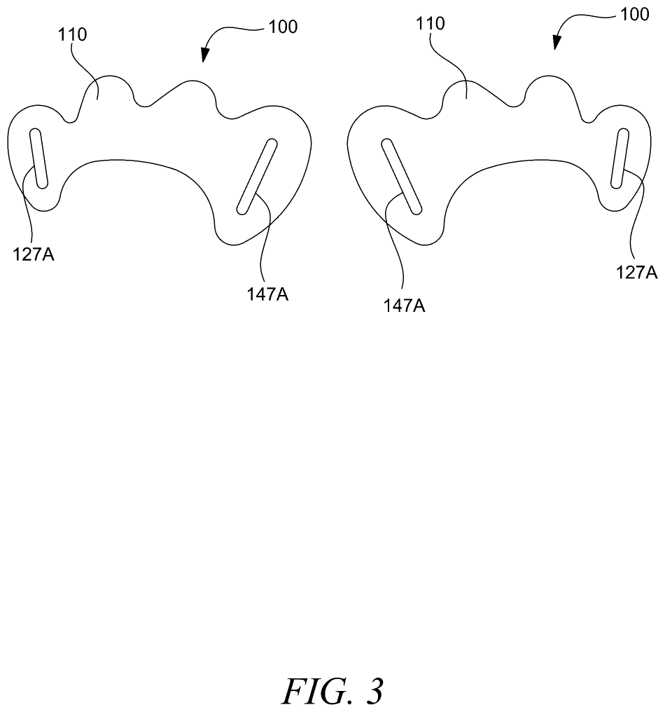

FIGS. 3-5 illustrate a top view, a front view, and a back view of a pair of adjustable toe spacer 100 respectively, according to an embodiment of the present invention. As seen in FIGS. 3-5, the pair of adjustable toe spacers 100 includes a left-foot toe spacer 100 and a right-foot toe spacer 100. The left foot toe spacer 100 and the right foot toe spacer 100 are mirror symmetric (mirror-image) to each other such that the left foot toe spacer 100 is suited for use on the left foot of the user and the right foot toe spacer 100 is suited for use on the right foot of the user.

FIG. 6 illustrates a top view of a pair of adjustable toe spacers 100 of FIGS. 3-5 being worn by the user. The pair of adjustable toe spacers 100 serves to prevent toe crowding by creating space between the toes of the user, helping restore the natural shape of the foot during use. The pair of base walls 110 and the plurality of spacer elements (120, 130, 140, 150) are integrally connected (formed) and made of styrene-ethylene-butylene-styrene (SEBS) gel. The advantage of using styrene-ethylene-butylene-styrene (SEBS) gel for manufacturing adjustable toe spacer 100 is that the adjustable toe spacer 100 is firm, yet flexible to provide a comfortable fit to various foot sizes.

In another embodiment, the pair of base walls 110 and the plurality of spacer elements (120, 130, 140, 150) are integrally connected (formed) and made of silicone gel.

In another embodiment, the pair of base walls 110 and the plurality of spacer elements (120, 130, 140, 150) are integrally connected (formed) and made of any flexible material such as but not limited to foam, silicone, gel, rubber, and combinations thereof.

FIG. 7 illustrates a top view of a pair of adjustable toe spacers 100 of FIGS. 3-5 being worn by the user, wherein a plurality of wedge inserts (128, 148) are removably inserted in slots (127, 147) of the adjustable toe spacer 100 of FIGS. 3-5. As seen in FIG. 7, a first wedge insert 128 is removably positioned in the first slot 127 through the slot opening 127A and a second wedge insert 148 is removably positioned in the second slot 147 through slot opening 127A. The plurality of wedge inserts (128, 148) enables the adjustable toe spacer 100 to change its size, thereby enabling customization of the adjustable toe spacer 100 according to the requirements of the user.

FIG. 8 illustrates a front perspective view of a plurality of wedge inserts (128, 148) of FIG. 7. The wedge inserts (128, 148) are substantially triangular-shaped (wedge-shaped) to enable easy manual insertion and removal in slots (127, 147) of the adjustable toe spacer 100. In an embodiment, the plurality of wedge inserts (128, 148) is made of Ethylene-vinyl acetate (EVA) foam. However, it should be understood that the plurality of wedge inserts (128, 148) could be made of any material such as but not limited to plastics, foam, rubber, metal, and combinations thereof.

In another embodiment (not shown in figures), the pair of base walls 110 and/or the plurality of spacer elements (120, 130, 140, 150) are made of a tearable (trimmable) material that is configured to be trimmed thinner for enabling use by a user with an overspread pinky toe. The user can cut a small portion of the pair of base walls 110 and/or the plurality of spacer elements (120, 130, 140, 150) of the adjustable toe spacer 100 in proximity of third hole 145 by using suitable common tools such as but not limited to: scissor, blade, knife and so on such that the adjustable toe spacer 100 could be used by persons with an overspread pinky toe, thus avoiding the need for using any special tools.

In another embodiment (not shown in figures), an adjustable toe spacer kit (not shown in figures) is described. The adjustable toe spacer kit (not shown in figures) comprises a pair of adjustable toe spacers 100, a plurality of wedge inserts (128, 148) that are pre-packaged in the form of a product and sold by supplier/retailer. The plurality of wedge inserts (128, 148) could include any arbitrary number of wedge inserts (128, 148), for instance, eight in number. The plurality of wedge inserts (128, 148) could have different sizes to adjust the spacing between toes according to the needs of the user.

The various components, and parts of the various embodiments of the adjustable toe spacer 100 of the present invention are similar and interchangeable. Further, it should be understood that the adjustable toe spacer 100 could be used for any application such as but not limited to yoga, exercising, barefoot training, relaxing, massaging, normal walking, running, and so on. Further, the user can use the adjustable toe spacer 100 while the user is already wearing footwear such as shoes, sandals, slippers, and so on.

Finally, while the present invention has been described above with reference to various exemplary embodiments, many changes, combinations, and modifications may be made to the exemplary embodiments without departing from the scope of the present invention. For example, the various components may be implemented in alternative ways. These alternatives can be suitably selected depending upon the particular application or in consideration of any number of factors associated with the operation of the device. In addition, the techniques described herein may be extended or modified for use with other types of devices. These and other changes or modifications are intended to be included within the scope of the present invention.

What is claimed is:

1. An adjustable toe spacer (100), comprising:
a pair of base walls (110) arranged substantially parallel to each other;
a plurality of spacer elements (120, 130, 140, 150) interconnecting the pair of base walls (110);
wherein the plurality of spacer elements (120, 130, 140, 150) and the pair of base walls (110) are connected to define a plurality of holes (125, 135, 145) that are dimensioned to receive a plurality of toes of a user; and
at least one slot (127, 147) that extends in a spacer element (120, 130, 140, 150) selected from the plurality of spacer elements (120, 130, 140, 150), wherein the slot(s) (127, 147) is configured to removably receive a wedge insert (128, 148) for adjusting the spacing between the plurality of toes of the user.

2. The adjustable toe spacer (100) according to claim 1, wherein the pair of base walls (110) and the plurality of spacer elements (120, 130, 140, 150) are integrally connected and made of styrene-ethylene-butylene-styrene (SEBS) gel.

3. The adjustable toe spacer (100) according to claim 1, wherein the pair of base walls (110) and the plurality of spacer elements (120, 130, 140, 150) are integrally connected and made of silicone gel.

4. The adjustable toe spacer (100) according to claim 1, wherein the pair of base walls (110) and the plurality of spacer elements (120, 130, 140, 150) are integrally connected and made of a flexible material selected from a group comprising of foam, silicone, gel or rubber and combinations thereof.

5. The adjustable toe spacer (100) according to claim 1, wherein the pair of base walls (110) and the plurality of spacer elements (120, 130, 140, 150) are made of a tearable material configured to be trimmed thinner for enabling use by a user with an overspread pinky toe.

6. The adjustable toe spacer (100) according to claim 1, wherein the plurality of spacer elements (120, 130, 140, 150) includes a first spacer element (120) measuring 0.35 inch in width, a second spacer element (130) measuring 0.12 inch in width, a third spacer element (140) measuring 0.12 inch in width, and a fourth spacer element (150) measuring 0.28 inch in width.

7. The adjustable toe spacer (100) according to claim 1, wherein the wedge insert (128, 148) is made of Ethylene-vinyl acetate (EVA) foam.

8. The adjustable toe spacer (100) according to claim 1, wherein the wedge insert (128, 148) is substantially triangular-shaped to enable easy insertion and removal in the slot(s) (127, 147).

9. The adjustable toe spacer (100) according to claim 1, wherein the plurality of spacer elements (120, 130, 140, 150) are four in number.

10. The adjustable toe spacer (100) according to claim 1, wherein the plurality of spacer elements (120, 130, 140, 150) are more than four in number.

11. The adjustable toe spacer (100) according to claim 1, wherein the plurality of holes (125, 135, 145) includes a total of three holes that are dimensioned to receive an index toe, a middle toe, and a ring toe of the user.

12. The adjustable toe spacer (100) according to claim 1, wherein the plurality of holes (125, 135, 145) includes more than three holes that are dimensioned to receive a big toe, an index toe, a middle toe, a ring toe, and a pinky toe of the user.

13. The adjustable toe spacer (100) according to claim 1, wherein the slot(s) (127, 147) is a through-hole.

14. The adjustable toe spacer (100) according to claim 1, wherein the slot(s) (127, 147) are two in number.

15. The adjustable toe spacer (100) according to claim 1, wherein the slot(s) (127, 147) are more than two in number.

16. The adjustable toe spacer (100) according to claim 1, wherein the slot(s) (127, 147) are less than two in number.

17. The adjustable toe spacer (100) according to claim 1, wherein each slot(s) (127, 147) includes a pair of slot openings (127A, 147A) and (127B, 147B) that are formed in the pair of base walls (110).
